(19) United States
(12) Patent Application Publication
(10) Pub. No.: US 2009/0311715 A1
(43) Pub. Date: Dec. 17, 2009

(54) IDENTIFYING CORONARY OR SOFT TISSUE CALCIFICATION

(75) Inventors: Whyte G. Owen, Rochester, MN (US); Virginia M. Miller, Rochester, MN (US); Muthuvel Jayachandran, Rochester, MN (US)

Correspondence Address: FISH & RICHARDSON P.C., PO BOX 1022, MINNEAPOLIS, MN, 55440-1022, US

(73) Assignee: Mayo Foundation For Medical Education and Research, Rochester, MN (US)

(21) Appl. No.: 12/435,734

(22) Filed: May 5, 2009

Related U.S. Application Data
(60) Provisional application No. 61/052,052, filed on May 9, 2008.

(51) Int. Cl. G01N 33/53

(52) U.S. Cl. 435/7.1

(57) ABSTRACT

This document relates to methods and materials involved in identifying calcification (e.g., coronary calcification or soft tissue calcification) in mammals and assessing thrombotic risk in mammals. For example, methods and materials involved in using microvesicles as a marker to determine whether or not a mammal (e.g., a human) has calcification or an elevated risk of thrombosis are provided. In addition, methods and materials for determining the amount and source of microvesicles are provided.

### CROSS-REFERENCE TO RELATED APPLICATIONS

This application claims the benefit of priority to U.S. Provisional Application Ser. No. 61/052,052, filed May 9, 2008.

### STATEMENT AS TO FEDERALLY SPONSORED RESEARCH

This invention was made with government support under grant number HL078638-01 awarded by the National Heart, Lung, and Blood Institute. The government has certain rights in the invention.

### BACKGROUND

1. Technical Field

This document relates to methods and materials involved in identifying calcification (e.g., coronary calcification or soft tissue calcification) in mammals and assessing thrombotic risk in mammals. For example, this document relates to methods and materials involved in using microvesicles as a marker to determine whether or not a mammal (e.g., a human) has calcification or an elevated risk of thrombosis.

2. Background Information

Atherosclerosis is considered an inflammatory disease involving lipidoses, hypertension, and platelet activation. Much of the current views of intravascular processes leading to formation of an arterial occlusion has been inferred from indirect analysis of circulating polypeptides, hormones, platelets, and leukocytes. Changes in circulating polypeptides are difficult to interpret as markers of intravascular processes as they turnover rapidly and their source cannot be identified. Likewise, circulating platelets and leukocytes have defined lifetimes and bear marks of interactions with each other or the vascular wall.

### SUMMARY

This document provides methods and materials for identifying calcification (e.g., coronary calcification or soft tissue calcification) in mammals and for assessing thrombotic risk in mammals. For example, this document provides methods and materials involved in using microvesicles as a marker to determine whether or not a mammal (e.g., a human) has soft tissue calcification or an elevated risk of thrombosis. This document also provides internal standards for assessing thrombotic risk in mammals.

As described herein, microvesicles can be used to identify a mammal as having soft tissue calcification. Having the ability to identify mammals with soft tissue calcification can allow clinicians to diagnose and treat cardiovascular conditions at earlier stages. In some cases, microvesicles can be used to identify a mammal at risk of developing thrombosis. Having the ability to identify mammals at risk of developing thrombosis can allow clinicians to start preventative therapies at earlier stages.

In general, one aspect of this document features a method for assessing a mammal for calcification. The method comprises, or consists essentially of, (a) determining whether or not plasma from the mammal contains an amount of microvesicles greater than about 1000 microvesicles per μL of the plasma, and (b) classifying the mammal as having calcification if the plasma contains the amount. The mammal can be human. The determining step can comprise using flow cytometry. The microvesicles can be platelet-derived microvesicles. The determining step can comprise determining whether or not the plasma from the mammal contains an amount of annexin-V positive microvesicles greater than about 1000 annexin-V positive microvesicles per μL of the plasma. The determining step can comprise determining whether or not the plasma from the mammal contains an amount of CD61 positive and annexin-V positive microvesicles greater than about 1000 CD61 positive and annexin-V positive microvesicles per μL of the plasma. The determining step can comprise determining whether or not the plasma from the mammal contains an amount of CD42a positive and annexin-V positive microvesicles greater than about 1000 CD42a positive and annexin-V positive microvesicles per μL of the plasma. The determining step can comprise determining whether or not the plasma from the mammal contains an amount of CD41 positive and annexin-V positive microvesicles greater than about 1000 CD41 positive and annexin-V positive microvesicles per μL of the plasma. The calcification can comprise coronary calcification. The calcification can comprise soft tissue calcification.

In another aspect, this document features a method for assessing a mammal for calcification. The method comprises, or consists essentially of, (a) determining whether or not plasma from the mammal contains an amount of endothelium-derived microvesicles greater than about 25 endothelium-derived microvesicles per μL of the plasma, and (b) classifying the mammal as having calcification if the plasma contains the amount. The mammal can be human. The determining step can comprise using flow cytometry. The determining step can comprise determining whether or not the plasma from the mammal contains an amount of annexin-V positive endothelium-derived microvesicles greater than about 25 annexin-V positive endothelium-derived microvesicles per μL of the plasma. The determining step can comprise determining whether or not the plasma from the mammal contains an amount of CD62-E positive and annexin-V positive endothelium-derived microvesicles greater than about 25 CD62-E positive and annexin-V positive endothelium-derived microvesicles per μL of the plasma. The determining step can comprise determining whether or not the plasma from the mammal contains an amount of CD146 positive and annexin-V positive endothelium-derived microvesicles greater than about 25 CD146 positive and annexin-V positive endothelium-derived microvesicles per μL of the plasma. The calcification can comprise coronary calcification. The calcification can comprise soft tissue calcification.

In another aspect, this document features a method for assessing a mammal for thrombotic risk. The method comprises, or consists essentially of, (a) determining whether or not plasma from the mammal contains an amount of microvesicles greater than about 1000 microvesicles per μL of the plasma or an amount of endothelium-derived microvesicles greater than about 25 endothelium-derived microvesicles per μL of the plasma, and (b) classifying the mammal as being at risk of developing thrombosis if the plasma contains either of the amounts. The method can further comprise determining the source of the microvesicles using an antibody. The antibody can be selected from the group consisting of anti-CD45, anti-CD33, anti-CD15, anti-CD56, anti-CD14, anti-CD3, anti-CD19, anti-CD20, anti-CD41, anti-CD61, anti-CD42a, anti-CD62-E, anti-CD146, and anti-glycophorin A antibodies.

In another aspect, this document features a method for determining the amount of microvesicles present in a sample. The method comprises, or consists essentially of, (a) adding a known amount of beads to the sample, and (b) using flow cytometry to detect microvesicles and the beads in the sample, thereby determining the amount of microvesicles present in the sample. The beads can have a 4.2 μm diameter. The method can further comprise adding 1 μm diameter beads and 2 μm diameter beads to the sample to aid in determining a flow cytometry size gate for the microvesicles. The sample can be a plasma sample obtained from a mammal using an anticoagulant. The microvesicles can be platelet-derived microvesicles. The microvesicles can be endothelium-derived microvesicles.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains representative scatter plot obtained by FACSCanto™ flow cytometry and representative electron micrographs. A: Control gates of buffer with fluorescein conjugated antibodies and calibration (size and True Count Beads™) beads in the absence of sample. B: Gates derived from adding a sample containing microvesicles to the buffer with fluorescein conjugated antibodies and calibration beads. C and D: Representative quadrants derived from the microvesicle gates shown in A and B, respectively. Counts were separated by antibody binding with Q3 representing microvesicles. E and F: Representative scanning (E) and transmission (F) electron micrographs of the isolated microvesicles. Arrow heads indicate membranes. G: Microvesicles imaged by CytoViva cell imaging system (dual fluorescence and optical microscopy) at 150×. H: Fluorescent beads (1 μM in diameter) imaged by the CytoViva cell imaging system at 150×.

FIG. 2 is graph plotting the analytical variability in total numbers of microvesicle between two aliquots of the same sample from individual women. Each point represents data from an individual woman.

FIG. 3 contains representative quadrants derived from flow cytometric scatter plots (upper panel) demonstrating annexin-V negative (Q3) and positive (Q4) microvesicles. Lower panel shows cumulative data of total (annexin-V negative (open bars), Q3, plus annexin-V positive (closed bars), Q4) microvesicles from women with negative (0 Agatston units, n=10) and positive coronary calcification scores (0.3-32 Agatston units, n=18 and 93-315 Agatston units, n=5). Data are presented as means±SEM. Asterisk denotes statistical significance (P<0.05) from those with zero calcium and calcium scores ranging from 0.3-32 Agatston units. FL1 represents green fluorescence. FL2 represents red fluorescence.

FIG. 4 contains representative quadrants derived from flow cytometric scatter plots of microvesicles labeled with platelet-specific antibodies CD61 (glycoprotein IIIa) (upper left panel) and CD42a (glycoprotein IX) (upper right panel) in combination with the antibody for annexin-V. Each quadrant was labeled to indicate positive staining. Cumulative numbers of CD61 (lower left panel) and CD42a (lower right panel) positive microvesicles from women with negative (0 Agatston units, n=10) and positive coronary calcification scores (0.3-32 Agatston units, n=18 and 93-315 Agatston units, n=5) are presented. Data are presented as means±SEM. Asterisk denotes statistical significance (P<0.05) from those with zero calcium and calcium scores ranging from 0.3-32 Agatston units.

FIG. 5 contains representative quadrants from flow cytometric scatter plot of microvesicles labeled to identify those derived from the endothelium by CD62-E (E-selectin) antibody and Annexin-V antibody (upper panel). Each quadrant is labeled to indicate separate populations of microvesicles. Cumulative data of CD62-E-PE positive microvesicles with (black bars) and without (open bar) annexin-V positive expression from women with negative (0 Agatston units, n=10) and positive coronary calcification scores (0.3-32 Agatston units, n=17 and 93-315 Agatston units, n=5) are presented. Data are presented as means±SEM. Asterisk denotes statistical significance (P<0.05) from those with zero calcium and calcium scores ranging from 0.3-32 Agatston units.

FIG. 6 is a bar graph of a prothrombinase assay of thrombin generation by microvesicles (10,000) derived from women with negative (0 Agatston units, n=10, closed diamonds) and positive coronary calcification scores (0.3-32 Agatston units, n=18, open squares, and 93-315 Agatston units, n=5, closed ovals). Open diamonds represent control for buffer with factors but no microvesicles (n=2). Data are presented as means±SEM.

### DETAILED DESCRIPTION

This document provides methods and materials related to identifying calcification (e.g., coronary calcification or soft tissue calcification) in mammals and assessing thrombotic risk in mammals. For example, this document provides methods and materials involved in using microvesicles as a marker to determine whether or not a mammal (e.g., a human) has soft tissue calcification or an elevated risk of thrombosis. As disclosed herein, if a sample (e.g., a plasma sample) from a mammal is observed to contain elevated levels of microvesicles (e.g., platelet-derived microvesicles), then the mammal can be classified as having a calcification or a risk of thrombosis. If a sample from a mammal is not observed to contain an elevated level of microvesicles, then the mammal can be classified as not having soft tissue calcification or a risk of thrombosis. Soft tissue calcification can include, without limitation, renal calcification, breast calcification, and calcification associated with ovarian disease. Thrombotic risk can include that associated with pregnancy and early stages of metastatic cancer.

Microvesicles, which circulate in the peripheral blood, are a heterogeneous population of spheres (vary in size from about 0.1 to 1.5 μm) formed from intact phospholipid rich membranes. Typically, a microvesicle contains at least half of the surface polypeptides, receptors, and lipids of their cells of origin. Microvesicles are differentiated from microparticles, the latter of which can refer to chemical particles or aggregates such as those formed from plasma lipoprotein or other chemicals. Microvesicles are smaller than platelets, which are typically between 2 and 2.5 μm in diameter, and are generated during cell activation and apoptosis induced by oxidative damage, inflammatory cytokines and chemokines, thrombin, bacterial lipopolysaccharide, shear stress, and hypoxia.

The term “elevated level” as used herein with respect to the level of microvesicles is any level that is greater than a control microvesicle level associated with normal, healthy mammals lacking calcification (e.g., coronary calcification or soft tissue calcification). In some cases, an elevated level of microvesicles can be a detectable level. In some cases, an elevated level of microvesicles can be any level that is greater than a reference level for microvesicles. The term “reference level” as used herein with respect to a microvesicle level is the level of microvesicles typically found in healthy mammals, for example, mammals free of soft tissue calcification and inflammatory disease. For example, a reference level of microvesicles can be the average level of microvesicles that is present in samples obtained from a random sampling of 50 healthy mammals matched for age and sex and free from environmental pollutants such as tobacco smoke. It will be appreciated that levels from comparable samples are used when determining whether or not a particular level is an elevated level.

An elevated level of microvesicles can be any level provided that the level is greater than a corresponding reference level for microvesicles. For example, an elevated level of platelet-derived microvesicles can be 0.5, 1, 2, 3, 4, 5, 6, 7, 8, 9, 10, 15, 20, or more times greater than the reference level for platelet-derived microvesicles. In addition, a reference level can be any amount. For example, a reference level for microvesicles can be zero. In this case, any level of microvesicles greater than zero would be an elevated level.

Any appropriate method can be used to determine the level of microvesicles present within a sample. For example, flow cytometry can be used to determine the level of microvesicles within a sample. Standards such as those described herein (e.g., beads of know size) can be used to identify and quantify microvesicles present within a sample. In some cases, the level of particular microvesicles (e.g., platelet-derived microvesicles, procoagulant microvesicles, or endothelium-derived microvesicles) can be determined using antibodies that can detect markers associated with particular cell types. For example, antibodies against the markers set forth in Table 1 can be used to identify and quantify particular microvesicles using flow cytometry techniques. In some case, anti-annexin-V antibodies or thrombin generation assays can be used to determine the level of procoagulant microvesicles present within a sample.

TABLE 1
Markers for identifying the source or type of microvesicles.

| Source or Type of Microvesicles | Markers |
|---|---|
| Procoagulant microvesicles | Annexin-V or thrombin |
| | generation assay |
| Leukocytes-derived microvesicles | CD45, CD11b |
| Granulocytes-derived microvesicles | CD33, CD15, CDllb; CD177 |
| NK cells-derived microvesicles | CD56 |
| Monocytes-derived microvesicles | CD14 |
| T-lymphocyte-derived microvesicles | CD3 and CD134 |
| B-lymphocyte-derived microvesicles | CD19 or CD20 |
| Platelet-derived microvesicles | CD41 or CD61 or CD42a |
| Endothelium-derived microvesicles | CD62-E, CD106, CD146 |
| Erythrocyte-derived microvesicles | Glycophorin A |

Any type of sample can be used to evaluate the level of microvesicles in a mammal including, without limitation, serum, blood, and plasma. In addition, any method can be used to obtain a sample. For example, a blood sample can be obtained by peripheral venipuncture. Once obtained, a sample can be manipulated prior to measuring the level of microvesicles. For example, a blood sample can be centrifuged to separate serum and plasma, and the separated serum and plasma can be liquid frozen for future analysis. Once obtained, the sample can be analyzed by flow cytometry based on size or using anti-marker antibodies to determine the total number of microvesicles or the level of particular types of microvesicles present within the sample.

In some cases, the methods and materials provided herein can be used to detect microvesicles generated in vivo from many cell types. A FACSCanto™ (New fourth or fifth generation) machine with high sensitivity and six colors detectors can be used to detect microvesicles. In some cases, blood can be prepared using anticoagulants as follows. The sample can be centrifuged (e.g., 3000 g for 15 minutes). The resulting supernatant from this spin can be removed and re-spun (e.g., using the same speed and duration). The absence of platelets from the supernatant of the second spin can be validated by Coulter counter. This platelet free plasma can be centrifuged (e.g., 20,000 g for 30 minutes), and the pellet can be washed (e.g., washed once with HEPES/Hanks buffer) and centrifuged (e.g., 20,000 g for 30 minutes) to prepare washed microvesicles. The supernatant can be discarded, and the pellet reconstituted with buffer (e.g., HEPES/Hanks buffer). These microvesicles can be stained to identify their cell of origin (all cells, not only platelets). Higher than 95% of microvesicles that circulate in the blood of healthy people can originate from platelets. Both buffers and antibodies can be filtered through 0.2 μm filters to remove contaminants before staining the isolated microvesicles. The signal to noise ratio can be high (e.g., 15-30,000 events:200-500 events). Scanning and transmission electron microscopy and Cyto viva can be used to verify the presence of microvesicles.

In some cases, the levels of microvesicles present within the sample can be used to diagnose patients with atrial fibrillation at risk for stroke. For example, a mammal with atrial fibrillation having an elevated level of activated endothelium-derived and/or platelet-derived microvesicles (e.g., annexin-V positive/CD62e positive microvesicles) can be diagnosed as at risk for stroke. In some cases, assessing the level of particular microvesicles (e.g., activated endothelium-derived microvesicles) can be used to monitor anti-coagulation treatments before, during, or after surgery. In some cases, assessing the level of particular microvesicles (e.g., activated endothelium-derived microvesicles) can be part of an annual physical as an indicator of disease risk (e.g., risk of soft tissue calcification, early atherosclerosis, or risk of venous or arterial thrombosis).

This document also provides methods and materials to assist medical or research professionals in determining whether or not a mammal has calcification or an elevated risk of thrombosis. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of microvesicles in a sample, and (2) communicating information about that level to that professional.

Any appropriate method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### EXAMPLES

### Example 1

### Association of Platelet and Endothelium-Derived Microvesicles with Coronary Artery Calcium

A cross-section study of a subset (n=33) of apparently healthy newly (between 6 mo and 3 years) menopausal women (n=146) screened to participate in a larger study was performed. The larger study was a randomized, placebo-controlled, double blinded, prospective multi-center trial evaluating women between the ages of 42-58 who are within three years of menopause to determine the effectiveness of oral and transdermal estrogen treatments to slow progression of carotid intimal thickness and coronary calcification. Because one exclusion criteria for the study was a coronary calcium score >50 Agatston units, all women undergo a coronary calcium scan at screening. Of the 146 women screened, five had an Agatston score >50 (range 93-315 Agatston units, eighteen had a coronary calcium scores >0 but <50 (range 0.3-32 Agatston units) and 123 had a score of 0 Agatston units. For this ancillary study, all women with a coronary artery calcium (CAC) score >0 were included and ten women randomly selected from the 123 with a CAC score=0 were also included. Among the 33 women included in this study, none was a current smoker, hypertensive, diabetic, or had a history of thrombotic disease.

Antibodies and Chemicals: Purified recombinant annexin-V-FITC (fluorescein isothiocynate, Cat. No. 556419), annexin-V-PE (R-phycoerythrin, Cat. No. 556421), mouse Ig fluorescence controls (mouse IgG1-FITC, Cat. No. 349041 and mouse IgG1-PE, Cat. No. 349043), R-PE-conjugated mouse anti-human CD11b/Mac-1 (Cat. No. 555388), CD14 (Cat. No. 340683), glycoprotein IX (CD42a, Cat. No. 558819), glycoprotein IIIa (CD61, Cat. No. 555754), CD62-E (E-selectin, Cat. No. 551145), CD56 (intercellular adhesion molecule-1, Cat. No. 555516), vascular cell adhesion molecule-1 (Cat. No. 555647) monoclonal antibodies and BD TruCOUNT beads were obtained from BD Biosciences, San Jose, Calif. Latex beads, 1 μm (Cat. No. L1030-1ML) and 2 μm (Cat. No. L0905-1 mL) amine modified polystyrene fluorescent yellow-green were obtained from Sigma (St. Louis, Mo.). Human factor Xa (Cat. No. HCXA-0060), factor Va (Cat. No. HCVA-0110), and prothrombin (Cat. No. HCP-0010) were obtained from Haematologic Technologies Inc. (Essex Junction, VT). Other reagents were analytical grade.

Coronary calcification: CAC images were obtained using a 64 detector CT scanner (Siemens Sensation 64; Siemens Medical Solutions, Forcheim, Germany) with a scan prescribed to cover the heart. Scans were electrocardiographically gated to the cardiac cycle, obtained in end inspiration using the following parameters: rotation time=0.33 seconds, collimation=24×1.2, with images reconstructed at 3 mm thick slices, pitch=0.2, kVP=120, and FOV=30 mAs was varied according to width of the patient on the scout image as measured across the liver. Patients who measured <32 cm had mAs=126. Patients who measured 32-38 cm had mAs=385. Patients who measured >38 cm had mAs=780. Images were reconstructed with a B35 kernel at 65% of the R-R interval.

The calcium score was calculated using commercially available semi-automated computer software (GE Smartscore, GE Healthcare, Milwaukee, Wis.) according to the Agatston method (Agatston et al., J. Am. Coll. Cardiol., 15(4):827-32 (1990)). Calcified plaque within each epicardial vessel was detected by four contiguous pixels and peak density >130 Hounsfield units (HU). The product of the area and respective density factor (1=130 to 199 HU; 2=200 to 299 HU; 3=300 to 399 HU, and 4 if ≧400) constituted the vessel-specific coronary artery calcium score. The calcium scores across each individual epicardial vessel were summed to determine the total coronary artery calcium score.

Blood sample collection: Venous blood samples were collected for determination of plasma lipids, hormones, high sensitivity C-reactive protein (hs-CRP), serum chemistries, and platelet functions. The anticoagulant used for each assay was dictated by the requirement of that assay. For microvesicle analyses, blood (4 mL) was collected through a 19 gauge butterfly needle by a trained phlebotomist into a tube containing 0.5 mL of 0.105 M sodium citrate and 0.1 mL of 1 M benzamidine hydrochloride, which anergized the platelets. Expression of P-selectin on the platelets under basal conditions and following stimulation with thrombin receptor agonist peptide (TRAP; 10 μM) was evaluated using standard techniques (Jayachandran et al., Appl. Physiol., 102(1):429-33 (2007)).

Blood Chemistries: Total cholesterol, low and high density lipoproteins, triglycerides, blood glucose, follicle stimulating hormone, estrogen, thyroid stimulating hormone, high sensitive C-reactive protein (hsCRP) in plasma and sodium, potassium, chloride, bicarbonate, creatinine, phosphorus, total protein, albumin, bilirubin, alkaline phosphatase, aspartate and alanine transaminases in serum and blood urea were measured. Total white blood cells, differential leukocytes, hemoglobin, and hematocrit were also determined. Platelet count was determined using a Coulter counter (T660).

Isolation of Blood Microvesicles: Plasma was prepared by centrifugation of the blood sample at 3000 g for 15 minutes within 30 minutes of the blood draw. Activated protein C was measured from this sample, and the remaining plasma was stored at −40° C. freezer for subsequent analysis. Plasma frozen at −40° C. was thawed in a 37° C. water bath for 5 minutes, vortexed, and centrifuged at 3000 g for 15 minutes. After centrifugation, 90% of the plasma supernatant was placed into a new tube and monitored by Coulter counter (platelet count ≦1) to confirm the absence of platelets and other cells from the sample. After validation, this plasma (0.5 mL) sample was then centrifuged at 20,000 g for 30 minutes. After centrifugation, plasma supernatant was again separated into another tube for storage at −80° C. for other analysis. Precipitant remaining from the 20,000 g centrifugation, which contained greater than 90% microvesicles, was reconstituted with 0.5 mL 20 mM HEPES/HANK'S/0.05% glucose buffer (pH 7.4), which was filtered twice through a 0.2 μm membrane filter. Tubes containing reconstituted microvesicles were vortexed and centrifuged again at 20,000 g for 30 minutes to remove plasma contaminations and to wash the isolated microvesicles. After centrifugation, the supernatant was discarded, and pelleted microvesicles were reconstituted again with 0.5 mL of above mentioned buffer and vortexed for 1 to 2 minutes. It was confirmed that recovery of microvesicles was the same from fresh and frozen plasma (Simak et al., Br. J. Haematol., 125(6):804-13 (2004) and Shet et al., Blood, 102(7):2678-83 (2003)).

Microscopic Observation of Isolated Microvesicles: For scanning electron microscopy, a small drop of isolated microvesicles from the 20000 g centrifugation was added to a parlodian-carbon coated grid and allowed to sit for 1 to 10 minutes. A pointed piece of filter paper was touched to the drop to remove excess liquid, leaving a thin film on the grid. A small drop of 1% phosphotungstic acid (PTA), pH 7.2, was added to the grid. The grid was then again touched by a pointed piece of filter paper to remove excess liquid, leaving a thin film on the grid which was allowed to air dry. If crystals from the buffer were evident, then the grid was washed by placing the grid in a drop of distilled water for 1 to 5 minutes. The grid was restained with PTA and then examined by scanning electron microscopy. For transmission electron microscopy, isolated microvesicles were placed in McDowell's and Trump's fixative for 1 hour or longer at 4° C. as described elsewhere (McDowell and Trump, Arch. Pathol. Lab. Med., 100(8):405-14 (1976)). After fixation, the fixed microvesicles were rinsed twice in 0.1 M sodium phosphate buffer (pH 7.2). Samples were then pelleted into liquid agar and then placed in 1% osmium tetroxide in the same buffer for 1 hour at room temperature. Samples were rinsed twice in distilled water followed by a 1-hour enbloc staining with 1% uranyl acetate. They were then dehydrated in an ethanolic series and embedded in quetol resin. Semi-thin (0.25-0.5 μm) sections were cut with glass knives and stained with 1% toluidine blue-O in 1% sodium borate. Ultrathin (70-90 nm) sections were cut with a diamond knife, stained with lead citrate and examined with a transmission electron microscope (FEI Technai-12 TEM, Hillsboro, Oreg.). The heterogeneous size of microvesicles isolated from fresh plasma was also confirmed by CytoViva dual mode fluorescence and optical microscopy cell imaging system (CytoViva, Inc. Auburn, Ala.).

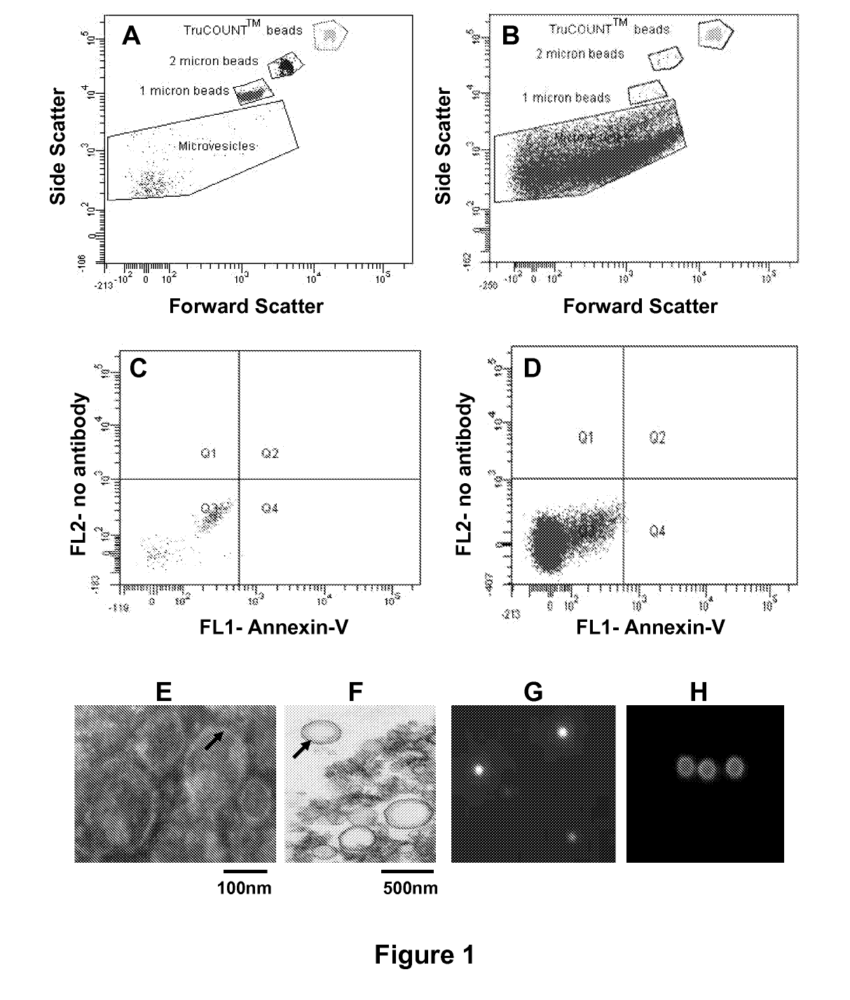

Identification of Isolated Microvesicles: Flow cytometry (FACSCanto™, BD Biosciences, San Jose, Calif.) was used to define microvesicles by size and positive fluorescence using marker specific antibodies. The gates to define size were set using an internal standard of 1 and 2 μm beads (TruCOUNT™, BD Biosciences, San Jose, Calif.). For quantification, samples were spiked with a known quantity of TruCOUNT™ beads of 4.2 μm diameter (FIG. 1). All buffers and antibodies were filtered twice through 0.2 μm filter to eliminate chemical particles and reduce instrument noise in the microvesicle gate. Fifty μL of isolated microvesicles were incubated with 4 μL of specified antibodies conjugated to fluroescein (Fl) isothioncynate and phycoerythrin for 30 minutes. After incubation, microvesicles were fixed with 400 μL of 1% paraformaldehyde for 15 minutes. After fixation, 50 μL of TruCOUNT™ beads were added immediately prior to analysis by flow cytometry.

Annexin-V binding as a measure of microvesicle surface phosphatidylserine was determined using annexin-V-FITC (BD Biosciences, San Jose, Calif.). Positively stained microvesicles can be separated by quadrant (Q1, Q2, Q3, Q4, as in FIG. 1). To validate that the fluorsceins did not affect microvesicle recovery, the annexin-V assay was repeated using phycoerythrin. Results were similar with each fluroscein. The absolute number of microvesicles was calculated from the number of events in the region containing microvesicles divided by the number of events in the calibration bead region times the number of beads (spiked known count) per test volume (FIG. 1). The same calculation was used to determine the absolute number of annexin-V positive or other cell specific marker-positive (or negative) microvesicles in each quadrant.

Non-specific annexin-V labeling (positive) was evaluated by preparing microvesicles in phosphate buffered saline (PBS) without calcium and then staining with annexin-V and analyzed FACSCanto™. No annexin-V positive events were observed in microvesicles prepared in PBS without calcium. Physiological concentrations of calcium is required for annexin-V binding, thus this experiment validates the specificity of the binding.

To determine intra-assay variability and whether measurements of total microvesicles were affected by a specific antibody, total numbers of microvesicles were measured in aliquots from the same sample incubated with four different antibodies.

Source (cells of origin) of Microvesicles: Platelet-derived microvesicles expressing phosphatidylserine were identified using mouse anti-human CD61 (glycoprotein IIIa)-pycoerythrin (CD61-PE) and CD42a (glycoprotein IX, CD42a-PE) antibodies with annexin-V-fluorescein isothiocyanate (FITC). Granulocyte-, monocyte-, and endothelium-derived microvesicles and phosphatidylserine expression were identified using mouse anti-human CD11b-PE, CD14-PE, CD62-E-PE antibodies with annexin-V-FITC, respectively. Microvesicles expressing cellular adhesion molecules such as inter-cellular adhesion molecule-1 (ICAM-1) and vascular cell adhesion molecule-1 (VCAM-1) were identified by ICAM-1-PE and VCAM-1-PE antibodies, respectively. All antibodies were from BD Biosciences (San Jose, Calif.), and the same lot number of each antibody was used for all assays reported herein.

Procoagulant Activity of Microvesicles: A prothrombinase activity assay was used to determine thrombin generating capacity of the microvesicles. For this assay, equal numbers (10,000) of microvesicles from each individual were incubated in Tyrode's buffer containing 5 nM Factor Xa and 10 nM Factor Va at 37° C. for 3 minutes. After incubation, 2 μM prothrombin and 50 μM fluorogenic substrate (D-VPR-ANSNH-C4H9·2 HCl) were added, and changes in fluorescence (thrombin generation) were measured immediately at 355 nM (excitation wave length) and 450 nM (emission wave length) for 30 seconds up to 3 minutes.

Data analyses: Data were presented as means±SEM. One-way analysis of variance (ANOVA) followed by Bonferroni's multiple comparison test was used to determine statistical significance. Correlation co-efficient was used to identify analytical variability of total number of microvesicles and activated microvesicles with the coronary calcification score. Statistically significance was accepted at p<0.05.

Characteristics of the study population: When women were grouped according to their CAC Agatston scores, there were no statistically significant differences in age, months past menopause, body weight, body mass index (BMI), lipid profile, follicle stimulating hormone, estrogen levels, or hsCRP levels among the groups (Table 2). Thyroid stimulating hormone was significantly greater in women with high calcium scores (93-315 Agatston units) compared to women with either moderate calcification (<35 Agatston units) or no calcification (Table 2). There were no statistically significant differences among the groups defined by coronary calcification in terms of serum chemistries (sodium, potassium, chloride, bicarbonate, creatinine, phosphorus, total protein, albumin, bilirubin, alkaline phosphatase, aspartate and alanine aminotransferases, blood urea and calcium), total, differential leukocytes, hemoglobin, hematocrit or platelet counts among groups; all were within normal range.

TABLE 2
Characteristics of women participating in this study.

| | Coronary Arterial Calcium Agatston Units | | |
|---|---|---|---|
| | | Moderate | High |
| | Negative | 0.3-35 | 93-315 |
| | (n = 10) | (n = 18) | (n = 5) |
| Age (years) | 53.1 ± 0.3 | 53.1 ± 0.4 | 54.8 ± 1.2 |
| Menopause (months) | 21.4 ± 2.9 | 16.8 ± 2.1 | 25.0 ± 3.8 |
| Body Weight (Kg) | 73.2 ± 3.3 | 75.6 ± 2.7 | 78.7 ± 7.3 |
| BMI | 26.1 ± 1.2 | 27.9 ± 1.0 | 28.6 ± 3.1 |
| Mean Coronary | 0 ± 0 | 8.4 ± 2.3† | 151.2 ± 41.3†* |
| Calcium(Agatston | | | |
| units) | | | |
| Mean Coronary | 0 ± 0 | 9.0 ± 1.8† | 133.3 ± 30.8†* |
| Calcium (volume) | | | |
| Total Cholesterol | 208.5 ± 11.3 | 213.2 ± 9.5 | 213.0 ± 12.6 |
| (mg/dL) | | | |
| LDL (mg/dL) | 125.8 ± 1.8 | 133.8 ± 6.9 | 120.6 ± 10.8 |
| HDL (mg/dL) | 2.8 ± 3.6 | 57.3 ± 4.4 | 59.0 ± 8.0 |
| Triglycerides (mg/dL) | 74.2 ± 11.9 | 114.3 ± 3.6 | 84.8 ± 38.3 |
| Blood Glucose | 91.0 ± 1.7 | 93.8 ± 2.3 | 102.4 ± 4.4 |
| (mg/dL) | | | |
| Follicle Stimulating | 83.4 ± 11.2 | 89.9 ± 9.5 | 74.2 ± 14.0 |
| Hormone (milliIU/L) | | | |
| Estrogen (pg/mL) | 22.4 ± 2.5 | 26.3 ± 5.0 | 49.6 ± 15.6 |
| Thyroid Stimulating | 2.2 ± 0.3 | 2.1 ± 0.2 | 4.5 ± 1.3†* |
| Hormone (milliIU/mL) | | | |
| High-sensitive | 2.0 ± 0.7 | 2.0 ± 0.4 | 1.5 ± 0.3 |
| C-Reactive | | | |
| Protein (mg/L) | | | |
| Framingham Risk | 1.1 ± 0.4 | 1.4 ± 0.2 | 1.8 ± 0.4 |
| Score | | | |

Values are shown as means + SEM;
*denotes statistically significant difference from women with Agatston scores of 0.3-35 and
†denotes statistically significant difference from women with Agatston scores of 0.

Validation of microvesicle isolation and assay: Expression of surface P-selectin on platelets, an indicator of platelet activation was <5% after collection (i.e. basal condition). Therefore, the procedure to collect the blood did not activate the platelets. Following activation with thrombin receptor agonist peptide (TRAP), expression of P-selectin increased to >90%. These results indicate that the platelets while not active in the basal state were viable, that is, they retained the ability to be activated by a platelet activators.

Microvesicle size was heterogeous, but all were less than 1 μm in diameter. The procedure used to isolate the microvesicles did not produce aggregates of microvesicles (FIG. 1).

The number of events measured by flow cytometry from filtered buffers and antibodies was low ranging between 100-250 events for a three-minute run (FIG. 1, panels A and C). However, more than 25,000 events were counted in the same buffer containing microvesicles (FIG. 1, panels B and D). These results indicate that events were due to the addition of microvesicles and not an artifact or impurities in the processing solutions.

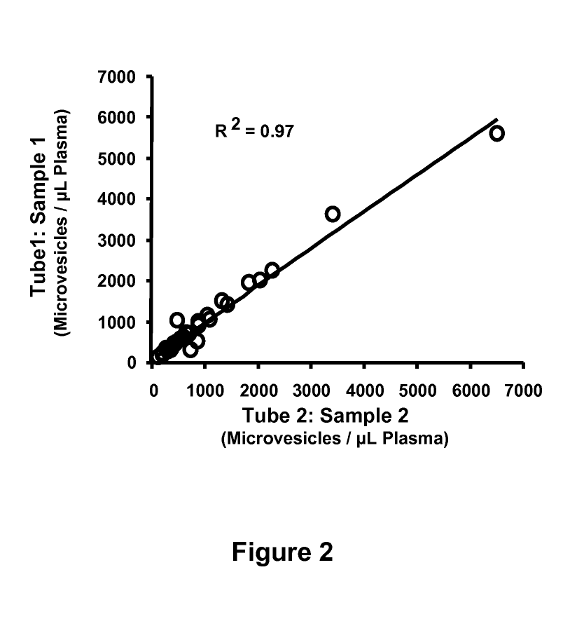

Total numbers of microvesicles (Q1+Q2+Q 3+Q4) detected by flow cytometry varied among individuals (FIG. 2). However, the ratio of the total number of microvesicles measured from a single individual but in two separate samples prepared with two different antibodies approached unity (FIG. 2). The co-efficient of variability calculated from a single sample measured with 4 different antibodies was 0.095. These results indicate that that intra-sample variability is small.

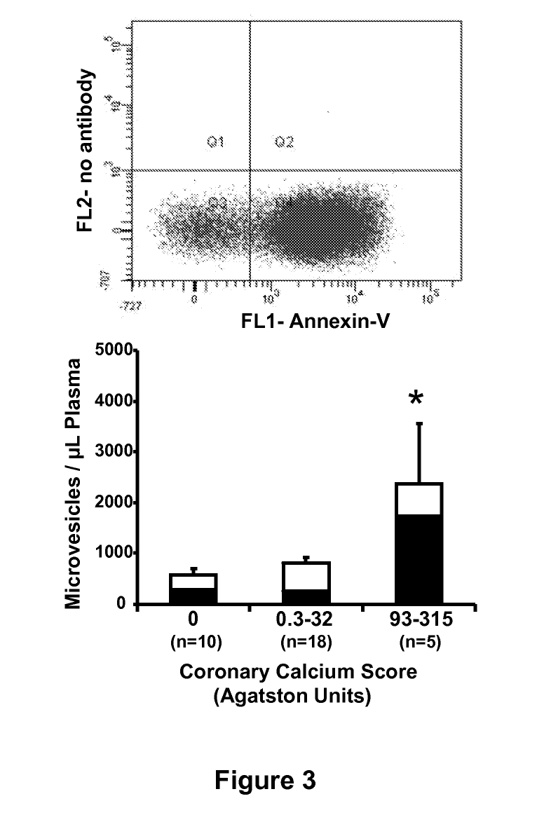

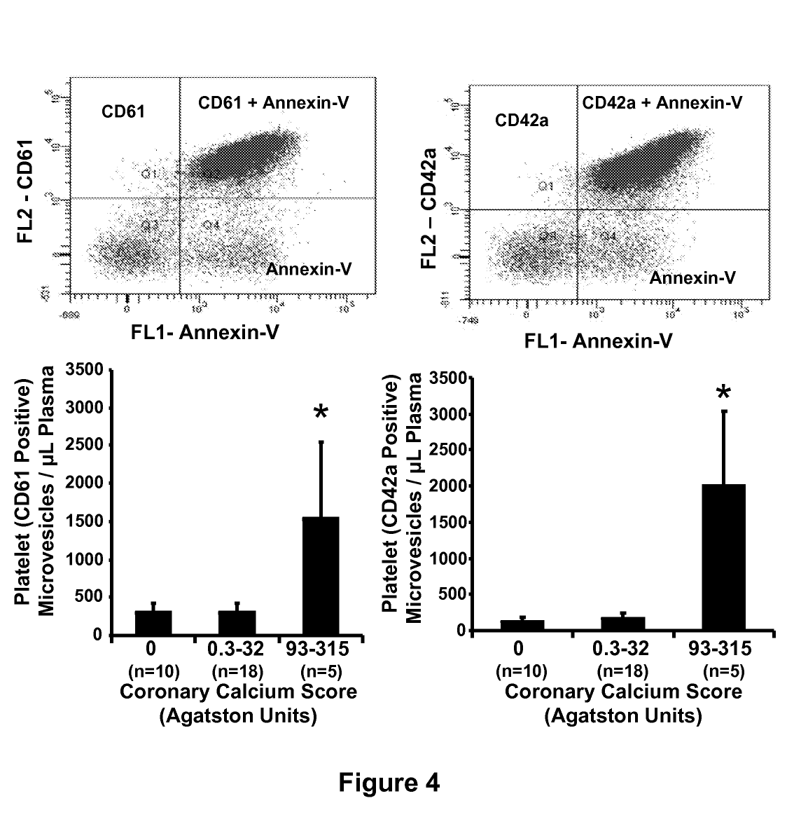

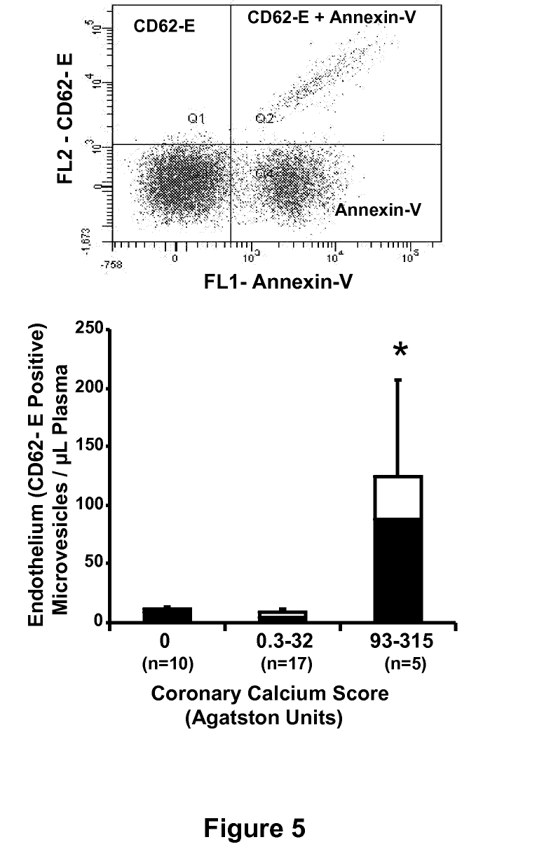

Relationship of microvesicles with CAC: Total number of microvesicles increased progressively with the CAC score (FIG. 2). In women with the highest calcium score, there were significantly more microvesicles positive for annexin-V than in the other two groups (FIG. 3). These annexin-V positive microvesicles in women with the highest calcium score were identified using two different platelet-specific antibodies (anti-CD61 (glycoprotein IIIa) and anti-CD42a (glycoprotein IX)) as being of platelet origin (FIG. 4). Endothelium-(CD62-E positive) but not leukocyte-(CD11b positive) and monocyte-(CD14 positive) derived microvesicles were also more prominent in women with the highest calcification scores (FIG. 5 and Table 3). ICAM-1 and VCAM-1 positive microvesicles did not differ among groups (Table 3).

TABLE 3
Coronary arterial calcium (CAC) scores and percent of marker positive
microvesicles.

| CAC | | | | | | | | |
|---|---|---|---|---|---|---|---|---|
| Scores | | | | | | | | |
| (Agatston | | CD11b + | | CD14 + | | ICAM-1 + | | VCAM-1 + |
| units) | CD11b | Annexin-V | CD14 | Annexin-V | ICAM-1 | Annexin-V | VCAM-1 | Annexin-V |
| Negative | 1.6 ± 0.8 | 7.3 ± 3.2 | 0.1 ± 0.0 | 0.8 ± 0.2 | 3.9 ± 1.5 | 11.9 ± 5.5 | 0.7 ± 0.2 | 2.6 ± 0.4 |
| (n = 10) | | | | | | | | |
| Moderate | 1.2 ± 0.5 | 3.3 ± 1.1 | 0.0 ± 0.0 | 0.7 ± 0.2 | 4.1 ± 0.9 | 14.9 ± 3.6 | 0.3 ± 0.1 | 1.9 ± 0.5 |
| 0.3-35 | | | | | | | | |
| (n = 18) | | | | | | | | |
| High | 1.7 ± 1.0 | 7.9 ± 3.5 | 0.0 ± 0.0 | 1.2 ± 0.6 | 3.6 ± 1.1 | 21.1 ± 6.8 | 0.1 ± 0.1 | 1.5 ± 0.7 |
| 93-315 | | | | | | | | |
| (n = 5) | | | | | | | | |

Data are presented Mean ± SEM of percentage of total microvesicles per μL plasma which expressed the marker.

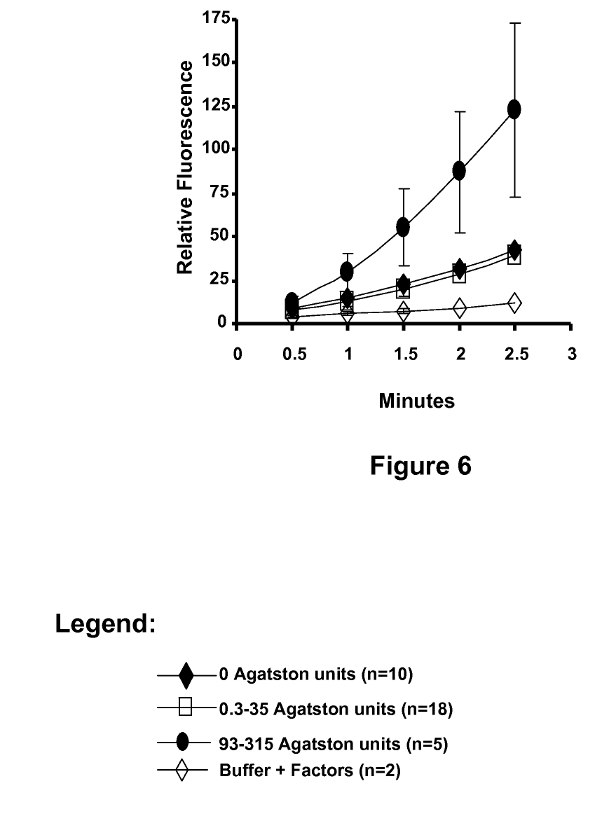

In vitro thrombin generation was proportional to the number of annexin-V positive microvesicles. The variability in thrombin generation was greatest in microvesicles derived from women with the high calcification scores (FIG. 6). These results indicate that there is a large difference in the range or level of cellular activation in individuals with ongoing disease processes associated with thrombotic risk with arterial calcification.

The results provided herein demonstrate the presence of coronary arterial calcification in about 16% of newly menopausal (3-36 months) women who would not otherwise be considered “at risk” for ASO based on plasma lipids, BMI, hs-CRP, triglycerides or other blood parameters, although there were non-significant trends for women with higher CAC Agatston scores to be heavier, with lower HDL cholesterol levels and higher fasting blood glucose values. The percentage of women positive for CAC was lower than that reported (about 50%) in women from the estrogen-only arm of the Women's Health Initiative. Although women in the estrogen-only arm of the WHI were within the same age range of the women in the present study, they were unlike women in the present study as those in the WHI had hysterectomies 10-20 years previously and thus were of between 5-10 years past surgical menopause. The present study documents CAC in women who are within three years of menopause.

The results provided herein also demonstrate that both the total number of microvesicles and those defined with an activated (phosphatidylserine positive) phenotype were highest in women with high CAC scores (>50 Agaston units). Furthermore, microvesicles expressing an activated phenotype were of platelet and endothelial origin.

The lack of correlation of coronary calcification with elevated total or LDL cholesterol or triglycerides, leukocyte-derived (granulocytes and monocytes) microvesicles, or inflammatory markers such as hs-CRP, ICAM-1 or VCAM-1 confirms data from other studies but is contrary to correlations of these markers with cardiovascular risk reported by others. Differences among studies emphasize that coronary calcification is a multi-factorial process and that, in some individuals, calcification may have an etiology independent of abnormalities in lipid metabolism. Alternatively, levels of calcification observed in these women were low and it is possible that microvesicles of other origins may be more prominent with calcification scores >500 Agastons units, or in individuals with elevated lipids, diabetes or kidney disease. It is important to emphasize that tests used in this study identified differences in circulating populations of microvesicles in women who did not have the usual cadre of risk factors for cardiovascular disease except for menopause.

While it is not possible from this study to establish a cause and effect relationship between platelet-endothelium-derived microvesicles and coronary calcification, these results suggest that monitoring origin and activation state of populations of microvesicles in the blood may allow for identification of a subset of women with early calcification and in whom follow-up screening or additional testing might be warranted.

While the results provided herein demonstrate the thrombin generating capacity is related to annexin-V positive microvesicles, these results extend previous observations to identify the population of annexin-V positive micorvesicles (phosphatidylserine-positive) as being of platelet and endothelial origin and that these are elevated specifically in women with coronary calcification. The thrombin generating capacity of microvesicles derived from women with the highest levels of calcification may define a procoagulant phenotype and identify those women at risk for enhanced thrombosis given a thrombin generating provocation. Thus, prediction of early cardiovascular risk and perhaps pre-mature coronary events in menopausal women could be improved by monitoring phosphatidylserine positive microvesicles of platelet and endothelial origin.

As reported by others, platelets are the main source of circulating microvesicles and express integrin αIIb/β3 (glycoprotein IIb/IIIa), platelet endothelial adhesion molecule (PCAM-1, CD31), P-selectin, CD63 (lysosomal glycoprotein III), and CD61 (glycoprotein IIIa). The tests used herein allow differentiation of platelet-derived microvesicles, which express phosphatidylserine. As platelet-derived microvesicles represent the major component of the total population of microvesicles, the ability to differentiate these populations of microvesicles can allow one to identify differences in the procoagulant or proinflammatory activity of the blood. Thus, it will be possible to evaluate how subpopulations of microvesicles change in a given individual with age and with hormone treatments.

The methods used herein to isolate microvesicles do not themselves generate microvesicles from circulating blood elements. For example, blood samples were collected under standard conditions into an anticoagulant that anergizes platelets. Furthermore, as P-selectin expression on platelets under basal conditions was low confirms that the platelets were not activated to generate microvesicles during the collection procedure. Variability in the number of microvesicles among individuals could result from impurities in the solutions and antibodies. However, the absence of counts in filtered solutions alone (FIG. 1) assures that counts obtained in solutions containing microvesicles were generated by the microvesicles and not by impurities in the test solutions. Variability in number of microvesicles from a single sample obtained from multiple readings was less than 10%, thus validating the reproducibility of the measurements. Another potential source of error in numbers of microvesicles may arise from the interaction of the microvesicles with the antibodies. However, numbers of microvesicles measured from a single sample but incubated with two different combinations of antibodies approached unity. Collectively, these validations steps assure that the association observed between CAC scores and microvesicle number, cells of origin, and thrombin generating capacity are not artifacts due to these assays.

In conclusion, the results provided herein demonstrate that coronary calcification as an expression of premature atherosclerosis can be detected in women who are within three years of menopause and who do not appear to be at high risk based on usual risk factors for coronary artery disease (low Framingham Risk Score). Furthermore, elevations in platelet- and endothelium-derived microvesicles provide new insight into early endothelial dysfunction associated with calcification. Changes in the circulating pool of microvesicles, which affect the thrombin generating capacity of the blood, can define a procoagulant phenotype for early menopausal women.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing a mammal for calcification, wherein said method comprises:
(a) determining whether or not plasma from said mammal contains an amount of microvesicles greater than about 1000 microvesicles per μL of said plasma or an amount of endothelium-derived microvesicles greater than about 25 endothelium-derived microvesicles per μL of said plasma, and
(b) classifying said mammal as having calcification if said plasma contains either of said amounts.

2. The method of claim 1, wherein said mammal is human.

3. The method of claim 1, wherein said determining step comprises using flow cytometry.

4. The method of claim 1, wherein said determining step comprises determining whether or not plasma from said mammal contains an amount of microvesicles greater than about 1000 microvesicles per μL of said plasma.

5. The method of claim 4, wherein said microvesicles are platelet-derived microvesicles.

6. The method of claim 4, wherein said determining step comprises determining whether or not said plasma from said mammal contains an amount of annexin-V positive microvesicles greater than about 1000 annexin-V positive microvesicles per μL of said plasma.

7. The method of claim 4, wherein said determining step comprises determining whether or not said plasma from said mammal contains an amount of CD61 positive and annexin-V positive microvesicles greater than about 1000 CD61 positive and annexin-V positive microvesicles per μL of said plasma.

8. The method of claim 4, wherein said determining step comprises determining whether or not said plasma from said mammal contains an amount of CD42a positive and annexin-V positive microvesicles greater than about 1000 CD42a positive and annexin-V positive microvesicles per μL of said plasma.

9. The method of claim 4, wherein said determining step comprises determining whether or not said plasma from said mammal contains an amount of CD41 positive and annexin-V positive microvesicles greater than about 1000 CD41 positive and annexin-V positive microvesicles per μL of said plasma.

10. The method of claim 1, wherein said determining step comprises determining whether or not plasma from said mammal contains an amount of endothelium-derived microvesicles greater than about 25 endothelium-derived microvesicles per μL of said plasma.

11. The method of claim 10, wherein said determining step comprises determining whether or not said plasma from said mammal contains an amount of annexin-V positive endothelium-derived microvesicles greater than about 25 annexin-V positive endothelium-derived microvesicles per μL of said plasma.

12. The method of claim 10, wherein said determining step comprises determining whether or not said plasma from said mammal contains an amount of CD62-E positive and annexin-V positive endothelium-derived microvesicles greater than about 25 CD62-E positive and annexin-V positive endothelium-derived microvesicles per μL of said plasma.

13. The method of claim 10, wherein said determining step comprises determining whether or not said plasma from said mammal contains an amount of CD146 positive and annexin-V positive endothelium-derived microvesicles greater than about 25 CD146 positive and annexin-V positive endothelium-derived microvesicles per μL of said plasma.

14. The method of claim 1, wherein said calcification comprises coronary calcification.

15. The method of claim 1, wherein said calcification comprises soft tissue calcification.

16. A method for assessing a mammal for thrombotic risk, wherein said method comprises:
(a) determining whether or not plasma from said mammal contains an amount of microvesicles greater than about 1000 microvesicles per μL of said plasma or an amount of endothelium-derived microvesicles greater than about 25 endothelium-derived microvesicles per μL of said plasma, and
(b) classifying said mammal as being at risk of developing thrombosis if said plasma contains either of said amounts.

17. The method of claim 16, wherein said method further comprises determining the source of said microvesicles using an antibody.

18. The method of claim 17, wherein said antibody is selected from the group consisting of anti-CD45, anti-CD33, anti-CD15, anti-CD56, anti-CD14, anti-CD3, anti-CD19, anti-CD20, anti-CD41, anti-CD61, anti-CD42a, anti-CD62-E, anti-CD146, and anti-glycophorin A antibodies.

19. A method for determining the amount of microvesicles present in a sample, wherein said method comprises:
(a) adding a known amount of beads to said sample, and
(b) using flow cytometry to detect microvesicles and said beads in said sample, thereby determining the amount of microvesicles present in said sample.

20. The method of claim 19, wherein said beads have a 4.2 μm diameter.

21. The method of claim 19, wherein said method further comprises adding 1 μm diameter beads and 2 μm diameter beads to said sample to aid in determining a flow cytometry size gate for said microvesicles.

22. The method of claim 19, wherein said sample is a plasma sample obtained from a mammal using an anticoagulant.

23. The method of claim 19, wherein said microvesicles are platelet-derived microvesicles.

24. The method of claim 19, wherein said microvesicles are endothelium-derived microvesicles.
